(19) United States
(12) Patent Application Publication
(10) Pub. No.: US 2019/0267122 A1
(43) Pub. Date: Aug. 29, 2019

(54) SYSTEMATIC PATIENT INFORMATION, RECORDS AND APPOINTMENT LIBRARY SYSTEM

(71) Applicant: GOPESH RAGHUVANSHI, NEW DELHI (IN)

(72) Inventor: GOPESH RAGHUVANSHI, NEW DELHI (IN)

(21) Appl. No.: 16/345,750

(22) PCT Filed: Oct. 27, 2017
(86) PCT No.: PCT/IB2017/056692

§ 371 Date: Apr. 29, 2019

(30) Foreign Application Priority Data
Oct. 27, 2016 (IN) 201611036813

(51) Int. Cl. G16H 20/10; G16H 80/00; G16H 10/60; G06Q 10/02

(52) U.S. Cl. CPC G16H 20/10; G06Q 10/02; G16H 10/60; G16H 80/00

(57) ABSTRACT

The present invention relates to a system and method to manage electronic prescriptions and other health related services including a health service provider appointment, retail pharmacy services, medical claim reimbursement, pathological services, etc. The system comprises a server module, a module to register at-least one user and/or a health service provider, a module to search at least one health service provider, a module to make an appointment with at-least one service provider of interest, a cloud database to store electronic prescriptions and related instructions, a payment gateway, and a module to transmit electronic prescriptions and related instructions to at-least one registered user and/or health service provider.

### FIELD OF THE INVENTION

The present invention relates to a system and method for electronically managing health services. More particularly, it relates to a system and method to manage Electronic Medical Records (EMR), in complete control of patient for keeping them secure. Other features linked to various health care services include, but not limited to retail pharmacy, medical claim, pathological services, Interfacing with third party activity trackers etc. on end to end basis, wherein the all the users are able to manage the services being taken or provided by them using single platform.

### BACKGROUND OF THE INVENTION

Managing Medical records correctly including pathological tests, diagnosis and prescription handling are essential steps in almost all health services wherein involvement of a health service provider is required. The prescription handling vis-à-vis management thereof becomes a painful job for a patient as well as to health service provider wherein, bulk prescription handling takes place. Further, it is difficult for a patient to manage all the treatment related prescriptions, reports, and invoices together at a same place.

Furthermore, a treatment procedure requires a mutual relationship of health service providers including doctors, clinics, hospitals, path-labs and pharmacies for easy interaction and creating a hassle-free situation for all the parties involved in a treatment procedure.

Sometimes, a patient has to move from one health service provider to another to take their services. This creates lots of trouble to patients to carry reports, diagnosis, prescriptions and other documents that needs to be reviewed by a service provider. For example: it is difficult and time consuming job for a patient to visit again to a pathologist to get a clinical report and take it again to the doctor's place for review purpose. Similarly, taking a prescription to a retail pharmacy store and keep waiting in a queue therein is a hectic job for a patient and their relatives as well.

There are disclosed various prescription management systems in the prior art. Some state of the art inventions relate to method and system for uploading/downloading patient data including state of health of a patient within a networked computer system. Another prior art teaches cloud based healthcare information exchange.

Publication US20020059082A1 suggests appointment setting and payment system. Similarly, US20050246200 provides for system, method, and computer program product for healthcare management.

Publication US20080319784 discloses an online health consultation method.

U.S. Pat. No. 5911687 describes a wide are medical information system and method of using the same. It provides a plurality of doctor terminals connected to the wide area network, a plurality of patient terminals connected to the wide area network, and at least one management server connected to the wide area network including an electronic case record file for storing at least clinic information for patients and a doctor database for storing data regarding a plurality doctors registered in the wide area medical information system.

US Pub. 20030018495 describe system and method for medical drug prescription acquisition. scheduling appointments with the doctor electronically through the Internet; filing and paying for a medical prescription online through the Internet; electronic notification of any events related to medical issues, e.g. change in the doctor's appointments, availability of prescriptions for pickup, etc.; doctor's online access to the patient's preferred drug list as specified by the patient's PBM and Health Insurance plan; the pharmacy's electronic receipt and confirmation of a patient's prescription via the Internet; the patient's electronic notification of the readiness of a filled prescription; and the simplifying the refilling of medical prescriptions.

All of the above cited art have disadvantages associated with them. For instance, none of the citation covers any system end to end. They need service provider on their network to use and provide services. Most of the citations are handling individual issues as appointment management, Prescription management or Pharma services. Also, the citations are silent on various aspects like providing for data privacy, emergency solutions, way-outs in casualty and/or emergent and urgent situations, pharmacy requirements and limitations, medical insurance liability as well as indemnities.

Moreover, there is a need of a platform that can assist users to do online monetary transactions for the health services provided in a healthcare system. Similarly, there is a need of a system that can integrate all the health care providers' including health insurance vendors on a single platform to transform the conventional way of delivering health services.

There is a need of an open architecture where service providers who are not part of health services network platform can also attend and facilitate patients who are part of such platform. There is required a system that connects all of the services on one platform to provide best services to a user while empowering a user to choose services for them in their desired manner.

Hence, there is a need of an approach to overcome all these problems by managing all health care services in an efficient manner.

### OBJECT OF THE INVENTION

The main object of the present invention is to provide an integrated platform to provide all possible health care services to patients in a well-defined network. Particularly, a solution for all medical needs across various medical practices like Allopathic, Homeopathic, Ayurveda, and Naturopathy etc.

Yet another object of the present invention is to provide a system and method for electronic prescription management.

Yet another object of the present invention is to provide a system to prepare an emergency profile of a user which can be used in case of any emergency situation.

Yet another object of the present invention is to provide a one touch SOS call to nearby Hospitals and Ambulance services with emergency profile details.

Yet another object of the present invention is to provide a system and method to manage health services including online appointment booking, retail pharmacy services, medical claim, and pathological services.

Yet another object of the present invention is to provide a system to approach and avail services of a service provider that is not part of the proposed network, and where the user securely shares their medical records with limited access rights to the service provider and only for one session.

Yet another object of the present invention is to provide a secured electronic medical record management under complete control of user for sharing or limited sharing of their medical records.

Yet another object of the present invention is to provide a system to record treatment history over a cloud database and keep updating patient's medical records automatically.

Yet another object of the present invention is to provide an approach to deliver electronic prescriptions to retail pharmacies for dispensing of medicines and consumer health related products.

Yet another object of the present invention is to provide a hassle-free health services management over a cloud database by providing multiple accesses to health services providers, and patients.

Yet another object of the present invention is to provide a system that includes a provision for one time registration of a user to maintain patient records accessible from any location over a cloud database.

Yet another object of the present invention is to provide a system that includes a provision to register health services providers for involvement and to deliver various health services to the registered users or patients.

Yet another object of the present invention to assist other health related services like medical claim reimbursements by linking at-least one policy with the registered account.

Yet another object of the present invention is to provide a system that allows a provision to link unique identification ID of a user to create an authentic and universal health service provider system.

### SUMMARY OF THE INVENTION

The present invention relates to a system and method to manage electronic prescriptions and other health related services including a health service provider appointment, retail pharmacy services, medical claim reimbursement, pathological services, etc.

The system comprises a server module, a module to register at-least one user and/or a health service provider, a module to search at-least one health service provider, a module to make an appointment with at-least one service provider of interest, a cloud database to store electronic prescriptions and related instructions, a payment gateway, and a module to transmit electronic prescriptions and related instructions to at-least one registered user and/or health service provider.

In an embodiment of the present invention, a method of managing electronic prescriptions and related information comprises the steps of:
registration of a user on the registration portal provided on a server;
creating or uploading a prescription(s) and/or treatment related data in an electronic media format on a cloud database by a registered user;
transmitting said electronic prescription(s) and/or treatment related data to a health service provider registered on the server; and
storing treatment history on the database over said cloud database.

In another embodiment, the present invention provides a method for managing health services, the method comprising the steps of:
exploring a registered medical service provider through a search module provided on a server;
selecting the service provider of interest;
booking an online appointment with the selected health service provider;
visiting the service provider's location to take health services;
uploading the treatment details including electronic prescriptions, laboratory reports, and related instructions on a cloud database provided on the server;
transmitting said treatment details to another health related service provider for further actions taken to assist a registered patient user; and
storing the overall treatment records for reference purposes.

### BRIEF DESCRIPTION OF THE DRAWINGS

A complete understanding of the system and method of the present invention may be obtained by reference to the following drawings:

FIG. 1a elucidates the system associated modules for respective users and services being provided under the process according to an embodiment of the invention;

FIG. 1b elucidates the patient module provided under the process according to the invention;

FIG. 1c elucidates the doctor module provided under the process according to the invention;

FIG. 1d elucidates the clinic module provided under the process according to the invention;

FIG. 1e elucidates the retail pharmacy module provided under the process according to the invention;

FIG. 1f elucidates the path lab module provided under the process according to the invention;

FIG. 2 elucidates the user registration process according to an embodiment of the invention;

FIG. 3 elucidates the appointment booking by a registered user under a process according to an embodiment of the invention;

FIGS. 4 and 5 elucidate the document uploading and updating by a registered user under a process according to an embodiment of the invention;

FIG. 6 elucidates the process of using the system by a registered user while visiting an unregistered service provider under a process according to an embodiment of the invention; and

FIG. 7 elucidates the data and information being transmitted and secured on cloud server.

### DETAILED DESCRIPTION OF THE INVENTION

Reference will now be made in detail to the present embodiment of the present invention, examples of which are illustrated in the description below. The embodiments are described below in order to explain the present invention.

In preferred embodiment, the present invention provides a system to manage electronic prescriptions and other health related services including a health service provider appointment, retail pharmacy services, medical claim reimbursement, pathological services, etc.

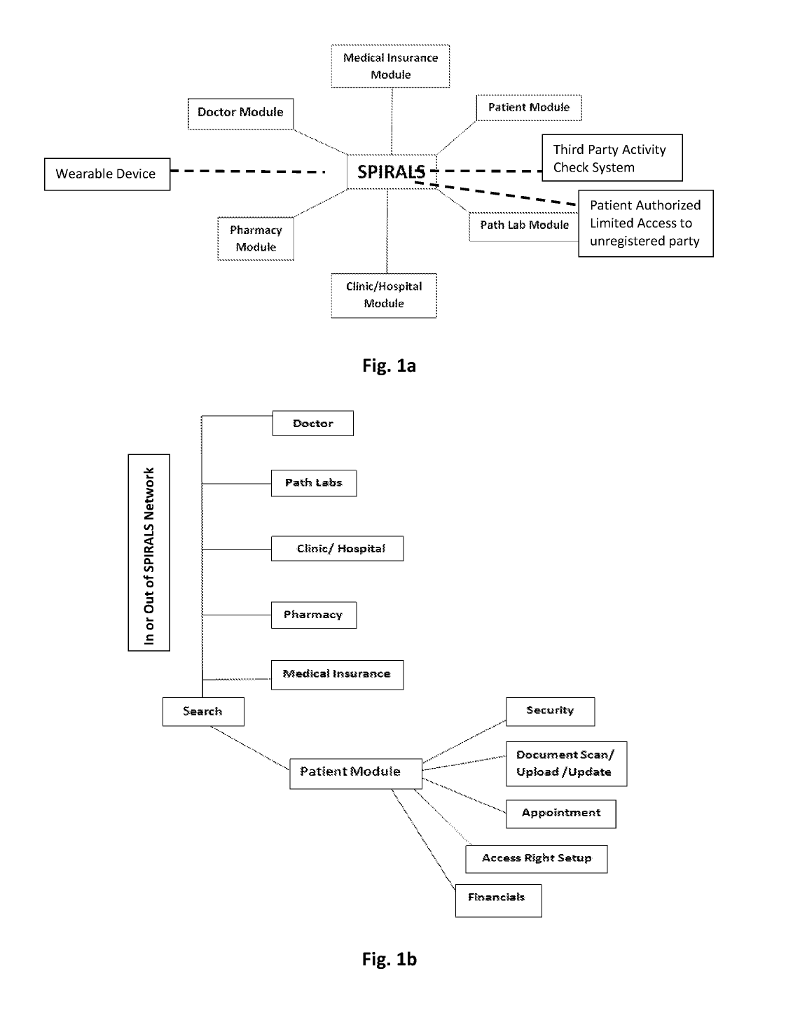

The system as depicted in FIG. 1a, comprises a server module, a module to register at least one user and/or a health service provider, a module to search at least one health service provider, a module to make an appointment with at least one service provider of interest, a cloud database to store electronic prescriptions and related instructions, and a module to transmit electronic prescriptions and related instructions to at least one registered user and/or health service provider. The system is accessible using specially enabled devices, wearable or otherwise. Further, a registered user can provide limited access under authorization to a non-network third party. Any non-network third party activity can be checked and scrutinized.

In FIGS. 1b to 1f, embodiments of the present invention are proposed plurality of user modules as described below:

Patient Module: Any person who is having his/her medical history and treatment going on. Even a doctor can have a patient profile in the system. This module would have patient related features and would provide dashboards to patient to analyze their health status.

Doctor Module: This platform won't restrict on any type of medical practitioner. Any doctor can register in the system. Search feature of the system would allow searching doctors based on the information provided by them.

Clinic/Hospital Module: Clinics and Hospitals can also take licenses of this system and provide to their doctors and lab to connect within this network. However, in this case these licenses would be transferable and can be moved to any other doctor within the clinic.

Pathological Lab Module: Path lab module would allow them to register and integrate themselves with this platform. Patients or doctors would be able to register appointments online and also receive their reports directly in their dashboards.

Pharmacy Module: This module would allow all registered patients and doctors and other users to request their medicines online. However, it would be required to have digitally signed prescriptions for such delivery OR pharmacy representative would need to check prescription while delivering medicines to the users.

Alternate Therapies Module: This module would allow Alternate therapists to register themselves like Physiotherapist, Dieticians, Specialized treatments like hair implants etc.

Insurance and Mediclaim Module: User should be able to link their Mediclaim and Insurance policies with them. User will also be able to search for various types of policies and Mediclaim available for them based on their profiles.

External Product Module: This module would allow integration of external modules like Activity trackers, Calorie counters etc. to keep track of any user's day activities. User's activities and data would automatically come to user dashboard and would give integrated view to the doctors.

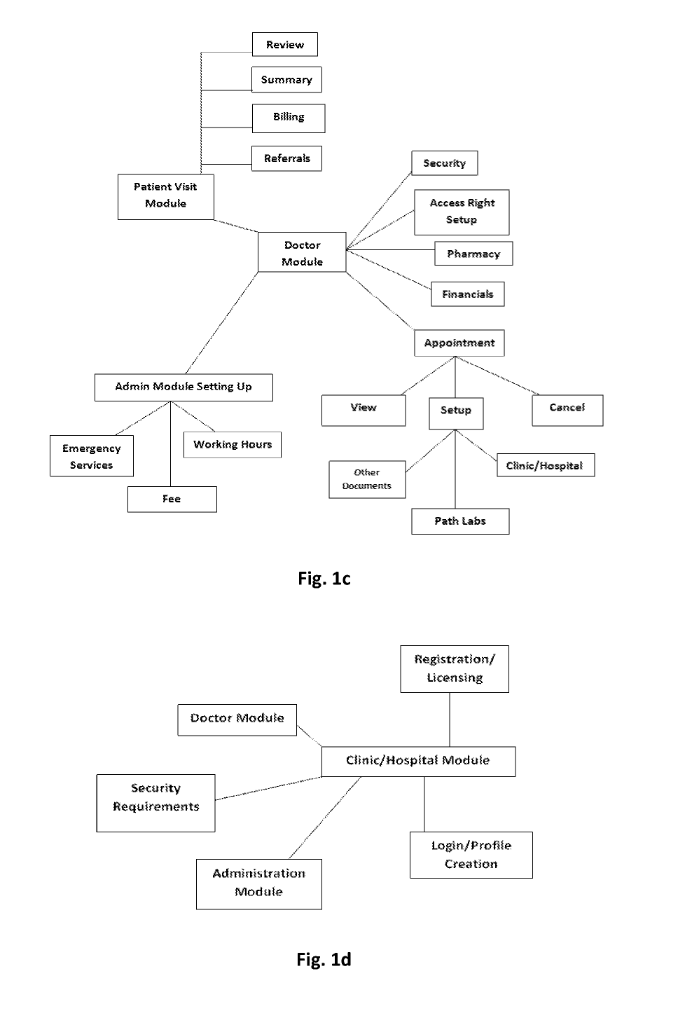

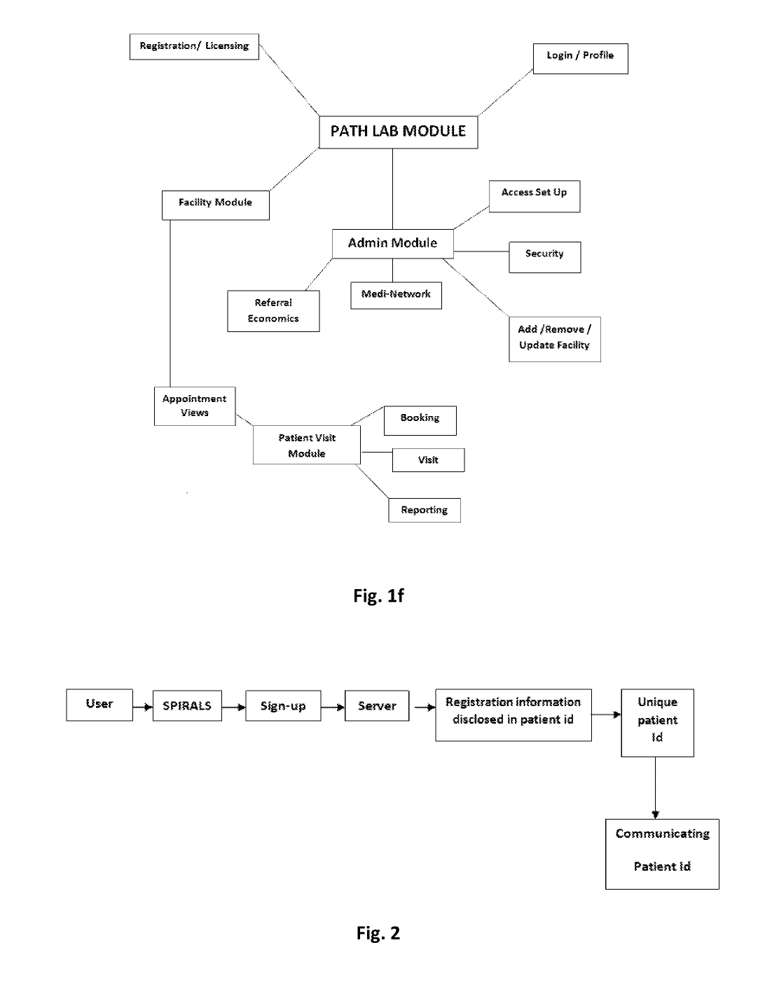

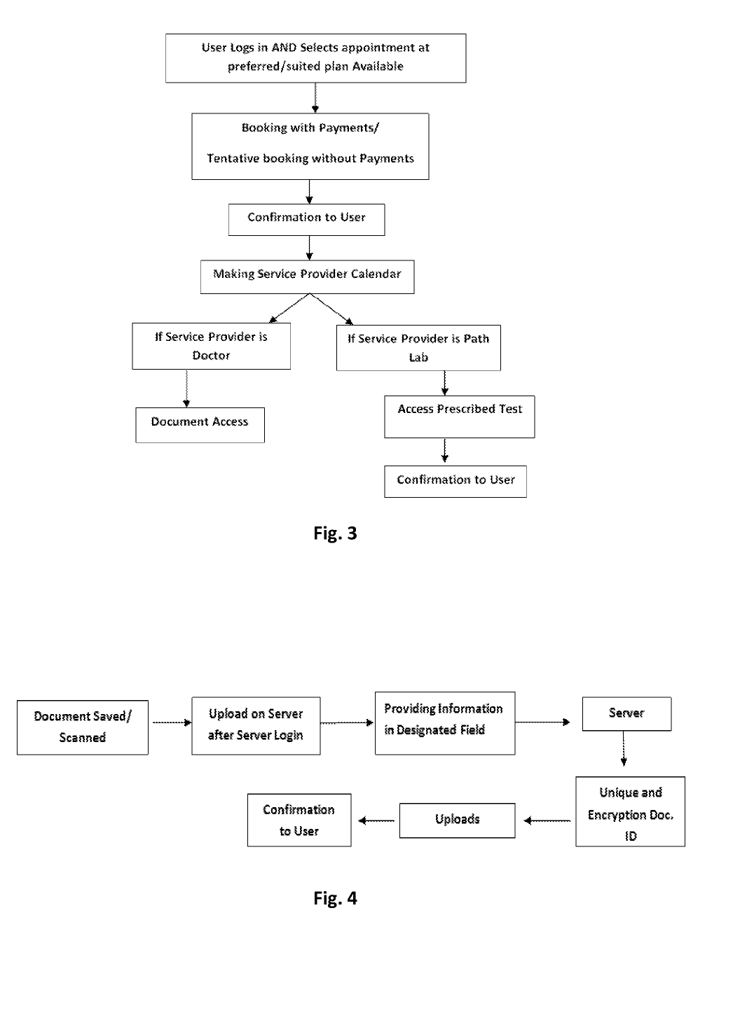

In another embodiment, a method of managing electronic prescriptions and related information, as shown in FIGS. 2 and 4, comprises the steps of:
registration of a user on the registration portal provided on a server;
creating or uploading a prescription(s) and/or treatment related data in an electronic media format on a cloud database by a registered user;
transmitting said electronic prescription(s) and/or treatment related data to a health service provider registered on the server; and
storing treatment history on the database over said cloud database.

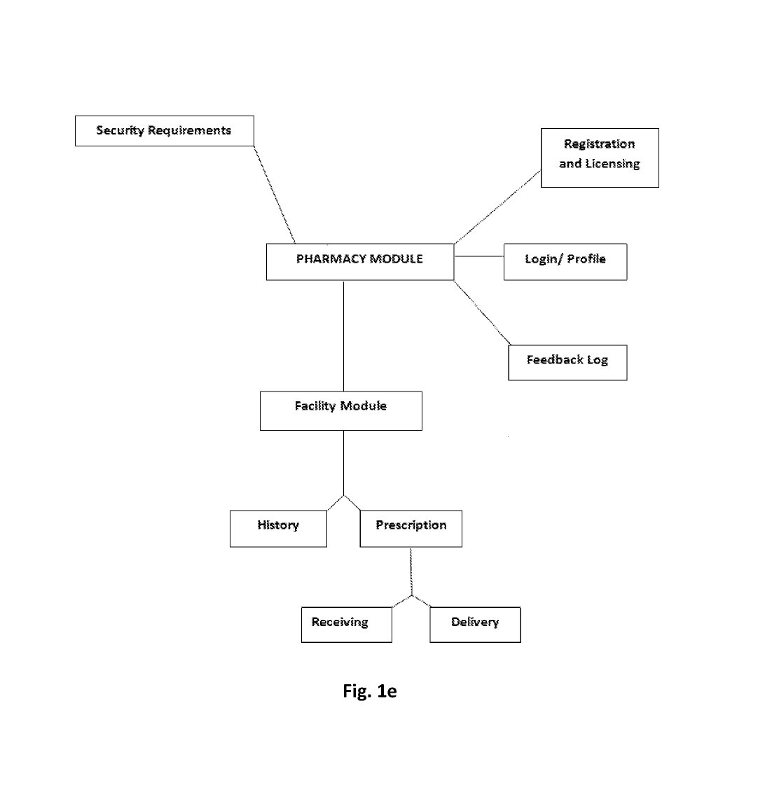

In another embodiment shown in FIG. 3, the present invention provides a method for managing health services comprising the steps of:
exploring a registered medical service provider through a search module provided on a server;
selecting the service provider of interest;
booking an online appointment with the selected health service provider;
visiting the service provider's location to take health services;
uploading the treatment details including electronic prescriptions, laboratory reports, and related instructions on a cloud database provided on the server;
transmitting said treatment details to another health related service provider for further actions taken to assist a registered patient user; and
storing the overall treatment records for reference purposes.

In another embodiment, the system provides a registration module to register at least one user to participate, manage, and interact with at least one module to manage health services and electronic prescriptions or other treatment related data.

In another embodiment, the user may be a patient user seeking a health service or the user may be a health service provider to assist a patient user in a treatment procedure.

After registering on the system, a registered user can search for at least one service provider of interest and can book an appointment with the selected health service provider(s). Furthermore, the system provides this search feature accessible to unregistered patient users to locate or search at least one health service provider of interest.

In case, a patient user has made an appointment with a doctor (a health service provider), the system may assist the patient user and doctor itself in many ways. After booking an appointment, the patient user has to visit the doctor's clinic for initial examination and treatment procedure, whatever the case it be. Thereafter, the doctor can advise or prescribe a suitable medication to said patient user directly on the proposed system by creating or uploading a prescription in an electronic format. In case the treatment demands for pathological or their clinical tests for examination purpose. The related instructions can be sent directly to a registered pathological service provider through transmission module of the proposed system. Similarly, the clinical reports can be uploaded to the corresponding account of the patient user for doctor's review and future reference purpose.

Likewise, the system integrates the retail pharmacy stores to provide their services to the registered users of the proposed system. The patient during a treatment procedure can get the prescribed medicines from the retail pharmacies based on latest prescriptions stored on the cloud database.

In this manner, the proposed system manages all these operations in a real time-manner. Furthermore, a backup can be created of an ongoing treatment procedure for future references.

Furthermore, the proposed invention provides security features to avoid any unwanted access into a registered account. For this purpose, a registered user may link a mobile phone number to activate an OTP (one time password) service for login purpose and to receive other notifications. The proposed system integrates at least one medical service provider to sell or to provide health insurance policies directly on the system. The registered user can search an insurance provider of interest and buy a policy directly from the system. Furthermore, a registered user can manage the policy and demand claims settlement directly from the system.

In another embodiment, the proposed system provides extra features including calorie-counter, diet management activities for users to keep a track on their daily activity. Furthermore, the user can ask service from a dietitian and maintain a diet plan accordingly.

In another embodiment, the proposed system includes a provision to manage emergency services by creating emergency accounts wherein, a rapid attention of health service providers is needed. For example: a road accident, or other medical emergency condition wherein a user can seek emergency service from a health service provider whenever required.

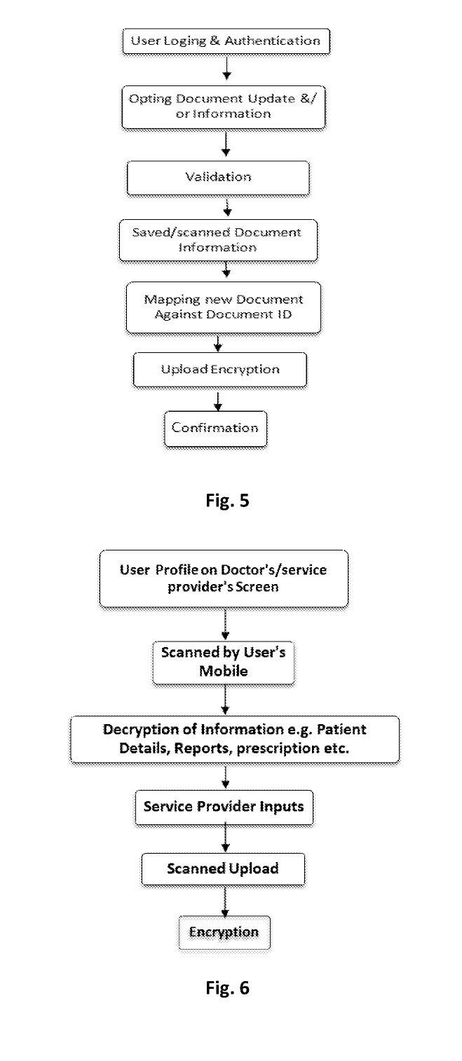

In another preferred embodiment of the present invention shown in FIG. 6, the patient data and medical history records are secured in encrypted form on remote server. With the intervention of patient in his presence, an unregistered health service provider can see them. In this embodiment, the QR code is generated on personal device of the patient which can be deciphered by patient by loging in into the health service provider's device.

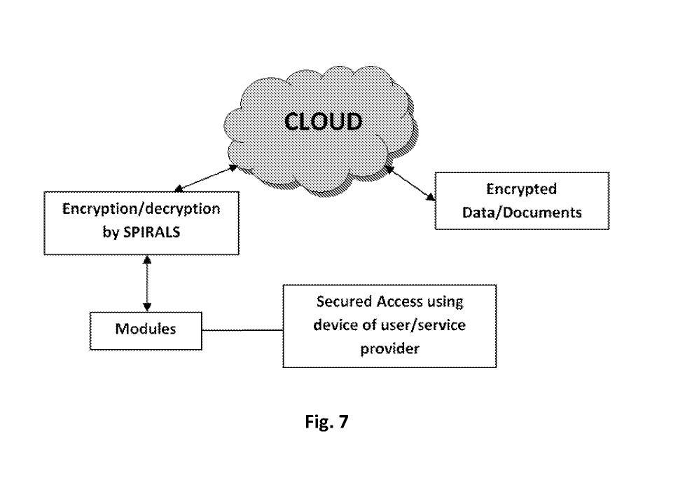

As shown in FIG. 7, the server module integrates a plurality of modules to manage various health services including management, transmission and storage of prescriptions and treatment related information in an electronic format. The server module integrates a cloud database for management and transmission purpose of prescriptions and other treatment related information.

In an embodiment of the present invention, patient would be able to create their emergency profile indicating ongoing treatments, allergies, family doctor contacts and emergency contact information. Further, an SOS feature is provided on request to users. It would be accessible from phone/tab in form of soft button AND will be provided as small wearable device. Pressing this soft button would activate user's mobile data, location services, would capture location details and send SOS message to Ambulance and Police services. And, same functionality would be achieved by just pressing that wearable device.

In another embodiment of the invention, monetary factors are tracked by the users. Since, user will be making all transactions through system, it would allow user to track their medical expenses and allow doctors to track their income due to appointments through system.

In an embodiment, every user will be linked with their Aadhar card number as one of their key identifier. This would allow user to be unique and would allow hospitals to get their information through Aadhar Card number.

I claim:

1. A method for electronically managing health services comprising the steps of:
a) registering at least one user and at least one health service provider;
b) exploring and selecting the registered health service provider by the registered user;
c) booking an appointment with the selected health service provider by the user with a unique booking code;
d) visiting and furnishing the unique booking code to the health service provider;
e) generating and uploading the treatment details in encrypted form on a cloud database provided on the server after visit by the health service provider or alternately by the user;
f) encrypting and storing all the details; and
g) further allowing decryption, transmittal and secured accession of the details;
wherein:
the unique booking code includes a QR code, generated on booking of an online appointment;
the method includes financial tracking of medical transactions by maintaining records of the transactions on services availed; and
the registered user and health service providers act as repositories that shall be linked to unique biometric ID for quick information access for any patient in an emergency situations or SOS situations.

2. The method as claimed in claim 1, wherein the step of registering a user includes personal, medical, insurance, identity, and allergy related details and wherein the step of registering a health service provider includes personal, identity and license & registration details.

3. The method as claimed in claim 1, wherein the health service provider includes but not limited to a doctor, a clinic, a health centre, a medical insurer, a retail pharmacy outlet, a pathological laboratory.

4. The method as claimed in claim 1, wherein selection of registered health service provider is based on the parameters of price, distance, availability, experience, number of years of experience, star rating of the previous users and parameters alike.

5. The method as claimed in claim 1, wherein the appointment status is selected from either tentative or confirmed and can be rescheduled.

6. The method as claimed in claim 1, wherein the step of transmittals and access includes at least one authentication step selected from but not limited to a PIN, a One Time Password, biometrics, multi-set password and alike.

7. The method as claimed in claim 1, wherein the payment for the appointment can be made online or on visit.

8. The method as claimed in claim 1, wherein the user can share his data temporarily with an unregistered health service provider using unique QR code for decryption of secured data.

9. A system for electronically managing health services comprising:
a) at least one storage server for storage of:
1) user data including registration details, prescription and/or treatment related data, insurance details, financial and transactional details; and
2) service provider data, including registration details, license data, facility details, financial and transactional details;
b) at least one communication server for transmitting the user data to a health service provider, sending notifications to the user and the health service provider, payment acknowledgement, appointment reminders, advice and directions to the user during prognosis, diagnosis, treatment and after treatment; and
c) at least one interface for exploring a registered health service provider through a search module provided on the storage server, making selection, booking an online appointment with the selected health service provider and receiving responses;
wherein:
the storage server is a cloud based server which stores data in encrypted form and is accessible using secure mode authentication including a PIN, a One Time Password, biometrics, multi-set password and alike;
the communication server transmits data towards and from the storage server in encrypted and decrypted forms respectively and is accessible using secure mode authentication including a PIN, a One Time Password, biometrics, multi-set password and alike; and
the interface for exploring a health service provider is accessible using secure mode authentication including a PIN, a One Time Password, biometrics, multi-set password and alike

10. The system as claimed in claim 9, wherein the health service provider includes but not limited to a doctor, a clinic, a health centre, a medical insurer, a retail pharmacy outlet, a pathological laboratory.
